SEQUENCE LISTING (3 sequences)

SEQ ID NO: 1 - protein, 11 aa, homo sapiens
ALQYKLEGTTR

SEQ ID NO: 2 - protein, 11 aa, homo sapiens
RLTRKRGLKLA

SEQ ID NO: 3 - protein, 11 aa, homo sapiens
ALSLSNKFVEG

(19) United States
(12) Patent Application Publication
(10) Pub. No.: US 2006/0148055 A1
(43) Pub. Date: Jul. 6, 2006

(54) Oxidised fragments of apolioprotein B and their uses

(76) Inventors: Joe Lunec, West Midlands (GB); Ruth Bevan, West Bridgford (GB); Helen Griffiths, Solihull (GB)

Correspondence Address: MONIQUE A. MORNEAULT, ESQ.;WALLENSTEIN WAGNER & ROCKEY, LTD., 311 South Wacker Drive - 5300, Chicago, IL, 60606, US

(21) Appl. No.: 11/294,972

(22) Filed: Dec. 6, 2005

Related U.S. Application Data
(63) Continuation-in-part of application No. 10/358,594, filed on Feb. 5, 2003.
Application No. 10/358,594 is a continuation of application No. 09/398,902, filed on Sep. 20, 1999, now abandoned.
Application No. 09/398,902 is a continuation-in-part of application No. PCT/GB98/00677, filed on Mar. 20, 1998.

(30) Foreign Application Priority Data
Mar. 20, 1997 (GB) 9705831.7

(51) Int. Cl. C07K 14/775; C12N 9/02

(52) U.S. Cl. 435/189; 530/359

(57) ABSTRACT

A molecule having the sequence of SEQ ID NO: 1 lysine 5 being conjugated with MDA is provided. The molecule inhibits uptake by the high affinity LDL receptor of LDL or a partially modified form thereof.

### CROSS-REFERENCE TO RELATED APPLICATIONS

This application is a Continuation-in-Part of co-pending application Ser. No. 10/358,594, filed on Feb. 5, 2003, which was a Continuation of application Ser. No. 09/398,902, filed on Sep. 20, 1999, now abandoned, which was a Continuation-in-Part of PCT/GB98/00677, filed Mar. 20, 1998, which claims priority from British Appln. No. 9705831.7, filed Mar. 20, 1997. The applications are commonly assigned and incorporated by reference herein.

### BACKGROUND OF THE INVENTION

Atherosclerosis is a major cause of mortality in all western populations. High serum cholesterol levels are associated with increased cardiovascular risk (Marmot, M., 1988, Atherosclerosis Reviews, 18: 95-108; WHO Monica Project, 1988, World Health Statistics Quarterly, 41: 115-138). However, oxidised sterols (cholesterol being a sterol) and not cholesterol per se appear to be the true agents which induce atherosclerotic lesions. Low levels of antioxidants, particularly vitamin E and B carotene are also associated with an increased risk of cardiovascular disease (Diplock, A. T., 1994, Mol. Asp. Med. 115: 295-376). These data suggest a role for the oxidation of lipids in the aetiopathogenesis of atherosclerosis.

Circulating cholesterol is contained primarily in apoprotein B—based low density lipoprotein (LDL)—a spheroidal particle comprising approximately 1500 cholesterol ester molecules surrounded by a layer of 800 phospholipid molecules, 500 cholesterol molecules and at least one 550 kDa molecule of apoprotein B100 (Apo B) (FIG. 1). Increased levels of LDL correlate strongly with accelerated atherosclerosis. Atherosclerosis is characterised by thickening and degeneration of the arterial intima, the pathogenesis falling into two defined stages—a first stage in which fatty streaks containing foam cells form in the intima, and a second stage in which fibrous plaques are formed within the artery.

Foam cells are formed from macrophages when oxidised LDL is endocytosed by the macrophages via a scavenger receptor. This fools the cell into believing that it has taken up too little cholesterol, causing cholesterol to enter the cell via the high affinity LDL receptors (which recognise predominantly the Apo B portion of LDL) in an uncontrolled manner (Steinberg, D. et al., 1989, N. Eng. J. Med., 320 (14): 915-924; Goldstein, J. L. et al., 1979, Proc. Natl. Acad. Sci. USA., 76 (1): 333-337) instead of its usual precisely controlled manner. This saturation of the macrophage with cholesterol results in its morphological change into a foam cell.

The oxidation of LDL results in the covalent binding of the oxidation products of the fatty acids on LDL with amino acid residues of the LDL proteins, including tryptophan, arginine, histidine and lysine, and resulting in the neutralisation of the positive charges on the amino acids (Esterbauer, H. et al., 1987, J. Lipid, Res., 28: 495-509; Chen. Q. et al., 1992, Biochem. J., 288: 249-254). Particular modification products of the fatty acids include malondialdehyde (MDA) (formed by the degradation of lipid peroxides) and 4-hydroxynonenal. Modification of LDL can be similarly achieved by glycation of Apo B (for example in poorly controlled diabetics), or by direct oxidation events. Tryptophan can also be modified to give N-formylkynurenine and bityrosine.

The present inventors have now identified peptide fragments of the apoprotein B 100 portion of LDL that are oxidised and present epitopes that prevent oxidised LDL from being uptaken by the scavenger receptor, thereby preventing the uptake of LDL by the high affinity LDL receptor.

### SUMMARY OF THE INVENTION

According to the present invention there is provided a molecule having the sequence ALQYKLEGTTR (SEQ ID NO: 1) or a partially modified form thereof or an analogue thereof, lysine 5 being conjugated with MDA, and which inhibits uptake by the high affinity LDL receptor of LDL or a partially modified form thereof. The sequence is residues 3349-3359 of apoprotein B 100 (full Apoprotein B sequence—Knolt, T. J. et al., 1986, Nature, 734-738), lysine 5 corresponding to residue 3353.

Also provided according to the present invention is a molecule having the sequence RLTRKRGLKLA (SEQ ID NO: 2) or a partially modified form thereof or an analogue thereof, lysine 5 being conjugated with MDA, and which inhibits uptake by the high affinity LDL receptor of LDL or a partially modified form thereof. The sequence is residues 3359-3369 of apoprotein B 100, lysine 5 corresponding to residue 3363

Also provided according to the present invention is a molecule having the sequence ALSLSNKFVEG (SEQ ID NO: 3) or a partially modified form thereof or an analogue thereof, lysine 7 being conjugated with MDA, and which inhibits uptake by the high affinity LDL receptor of LDL or a partially modified form thereof. The sequence is residues 3371-3381 of apoprotein B 100, lysine 7 corresponding to residue 3377.

Partial modification of the amino acid sequence may be by way of addition, deletion or substitution of amino acid residues or by other chemical modification, the partially modified molecule inhibiting uptake of LDL by the high affinity LDL receptor. Analogues (for example mimitopes) of the amino acid sequences and the epitopes displayed may be readily produced (Geysen, H. M. et al., 1987, Journal of Immunological Methods, 102: 259-274), the analogues inhibiting uptake by the high affinity LDL receptor of LDL or a partially modified form thereof. Partially modified sequences may be homologues of the sequences from which they were derived.

The conjugated MDA may be either MDA itself or a closely related derivative of MDA, for example an α,β unsaturated aldehyde derivative of MDA.

Modification of LDL may for example be by conjugation with MDA, by glycation or by conjugation with hydroxy alkenals such as 4-hydroxynonenal.

Molecules according to the present invention may be for use as immunogens, e.g. for the production of antibodies (or antigen binding fragments thereof) against them (Harlow, E. and Lane, D., “Antibodies—A Laboratory Manual”, Cold Spring Harbor Laboratory, Cold Spring Harbor Press, N.Y., 1988). The term “antibody” is used to describe any antigen-binding species, for example an antigen-binding antibody fragment.

The molecules according to the present invention may be for use in a method of treatment or diagnosis of the human or animal body. Particularly, they may be for the treatment of diagnosis of atherosclerosis. Treatment may of course be both prophylactic and therapeutic.

Also provided according to the present invention is the use of a molecule according to the present invention in the manufacture of a medicament for inhibiting uptake by the high affinity LDL receptor of LDL or a partially modified form thereof. Also provided is a method of manufacture of same medicament, comprising the use of a molecule according to the present invention.

Methods of manufacture are well known in the art. For example, a molecule according to the present invention may be provided with a pharmaceutically acceptable carrier, diluent or excipient (Remington's Pharmaceutical Sciences and US Pharmacopeia, 1984, Mack Publishing Company, Easton, Pa., USA), ready for e.g. intra-venous injection. Similarly, the dose to give may be readily determined using standard in vitro/in vivo does-response experiments. Culture systems for macrophages are well known and may be readily employed in such experiments.

Also provided according to the present invention is antibody which binds specifically with the molecules of the present invention. Also provided is antibody which binds specifically with the molecules of the present invention for use in a method of treatment or diagnosis of the human or animal body for example detection of modified or conjugated LDL or peptides of same. Molecules, antibodies or antigen binding fragments thereof may also be for immunotherapeutic use. Binding agents other than antibodies, which agents bind specifically to the molecules of the present invention may equally be used.

Also provided according to the present invention is the use of antibody according to the present invention in the manufacture of a medicament for inhibiting uptake by the high affinity LDL receptor of LDL or a partially modified form thereof. Also provided is a method of manufacture of same medicament, comprising the use of antibody according to the present invention. A particular use of the antibody is in the treatment or diagnosis of atherosclerosis, treatment being both prophylactic and therapeutic.

Also provided according to the present invention is the use of a molecule according to the present invention in a diagnostic test method for antibody specific against oxidised LDL which will cause uptake by the high affinity LDL receptor of LDL or a partially modified form thereof. Also provided is the use of antibody according to the present invention in a diagnostic test method for oxidised LDL which will cause uptake by the high affinity LDL receptor of LDL or a partially modified for thereof.

Also provided is a diagnostic test method for oxidised LDL which will cause uptake by the high affinity LDL receipt of LDL or a partially modified form thereof, comprising the steps of:

i) reacting an antibody according to the presention invention with a sample;

ii) detecting an antibody-antigen binding reaction; and

iii) correlating detection of the antibody-antigen binding reaction with the presence of oxidised LDL which will cause uptake by the high affinity LDL receptor of LDL or a partially modified form thereof.

Also provided is a diagnostic test method for antibody specific against oxidised LDL which will cause uptake by the high affinity LDL receptor of LDL or a partially modified form thereof, comprising the steps of:

i) reacting a molecule according to the present invention with a sample;

ii) detecting an antibody-antigen binding reaction; and

iii) correlating detection of the antibody-antigen binding reaction with the presence of antibody specific against oxidised LDL which will cause uptake by the high affinity LDL receptor of LDL or a partially modified form thereof.

Also provided according to the present invention is a diagnostic test method for modified or conjugated LDL or peptides of the same which will not be taken up by the high affinity of LDL receptor, comprising the steps of:

i) reacting an antibody or antigen binding fragment specific to a molecule according to the present invention with a sample;

ii) detecting an antibody-antigen binding reaction; and

iii) correlating detection of the antibody-antigen binding reaction with the presence of oxidised or conjugated LDL or peptides of the same which will not be taken up by the high affinity LDL receptor.

The sample may be a patient plasma sample.

The plasma may contain antigen representing various types of oxidation and conjugation.

Molecules and antibody according to the present invention may be used to quantitatively standardise results obtained from such diagnostic tests, or may be used as reagents in the tests. They may have therapeutic benefit as antagonists and could prevent foam cell formation.

Diagnostic test methods may include ELISA (enzyme-linked immunosorbent assay), for example antigen capture ELISA or competitive ELISA, immunoturbidimetry or dip-stick assays (WO 88/08534).

In order to test for specific conditions and causes of oxidation of LDL (for example oxidation caused by hyperglycaemia, hypercholesterolaemia, systemic lupus erythematosis (SLE) or hereditary conditions, molecules according to the present invention modified by for example glycation or conjugation with MDA may be used as appropriate, as may antibody specific to the molecules.

Also provided according to the present invention is a diagnostic test kit for performing a diagnostic test method according to the present invention. Such a kit may include instructions for its use (i.e. for performing an appropriate diagnostic test method according to the present invention).

The invention will be further apparent from the following description, with reference to the several figures of the accompanying drawings, which show, by way of example only, methods of detection of oxidised or conjugated LDL and of peptides of same.

### BRIEF DESCRIPTION OF THE DRAWINGS

FIG. 1 shows a schematic representation of an LDL particle and Apo B epitopes. The location of Apo B fragment responsible for LDL binding to the high affinity LDL receptor is shown as the amino acid residues 3441-3569 defined by monoclonal antibody Mb47 (Knolt, T. J. et al., supra). Mb47 blocks LDL binding to the high affinity LDL receptor. T₂T₃ suggests the boundary of thrombolytic peptides;

FIG. 2 shows the common mechanism of modification of LDL through glycation and conjugation with MDA;

FIG. 3 shows the conjugation of MDA with lysine;

FIG. 4 shows a comparison of peptide ALQYKLEGTTR (SEQ ID NO: 1) before (a) and after (b) conjugation with MDA. X axis shows the mass/charge ratio; Y axis shows relative abundance. Numbered peaks are at (FIG. 4a) 1355.09 and 1425.43 and (FIG. 4b) at 1483.16, 1504.31 and 1609.16 on the X-axis;

FIG. 5 shows a mass spectrometry analysis of the peptide of SEQ. ID NO. 1 conjugated to MDA following typtic digestion; and

FIG. 6 shows a graph comparing the effects of peptide competitors on ox LDL uptake by scavenger receptors.

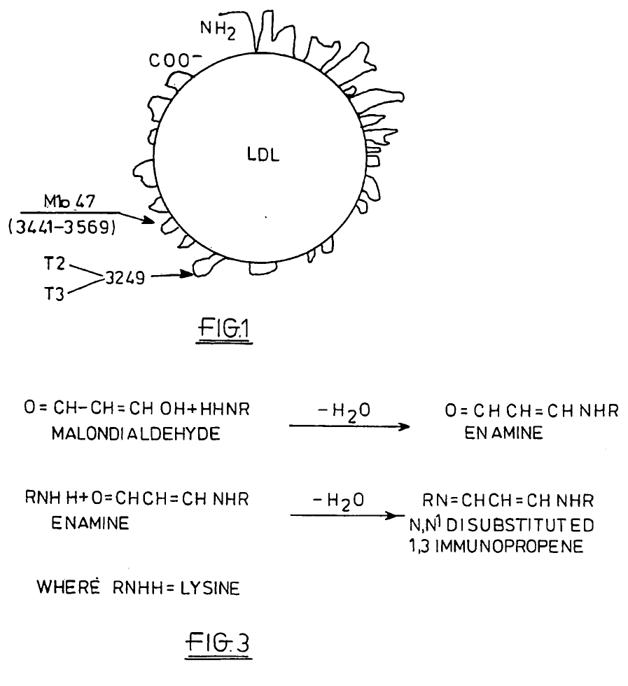

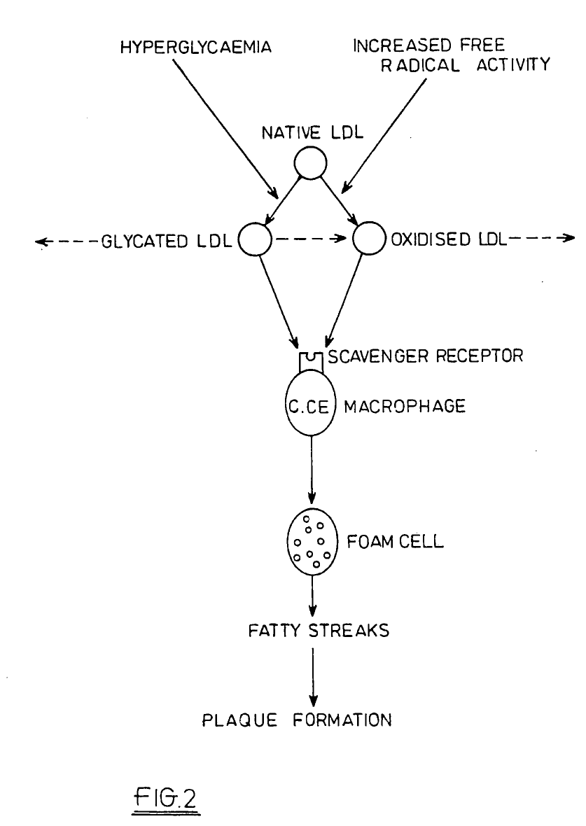

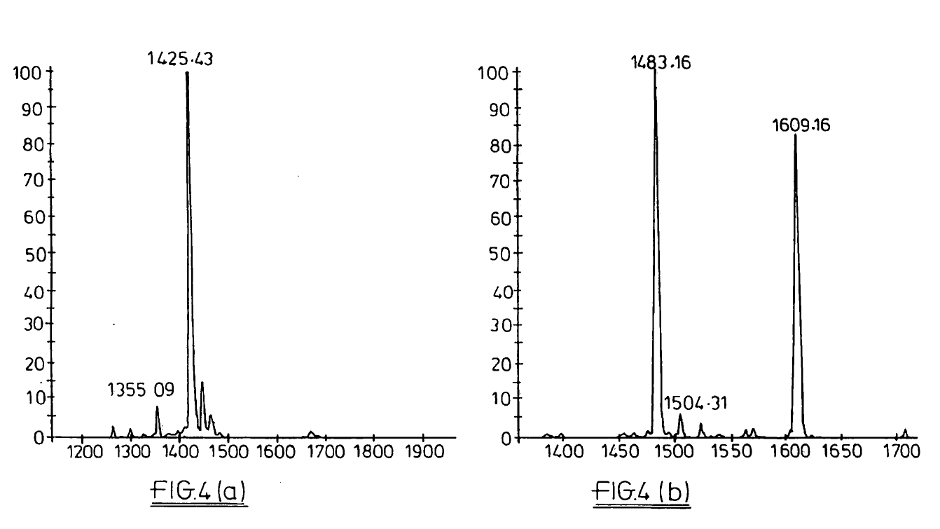

### DETAILED DESCRIPTION

### Example 1

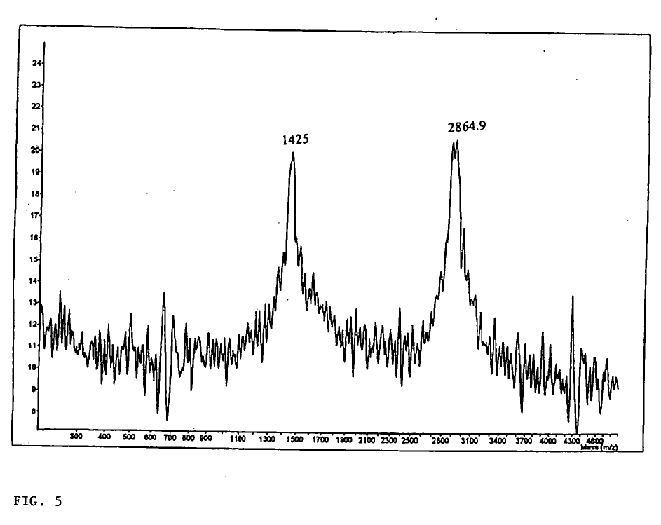

The peptide sequence ALQYKLEGTTR (SEQ ID NO: 1) was synthesised using standard methods and conjugated with MDA. FIG. 4 shows the results of a mass spectrometry plot of the peptide before and after conjugation. Molecular weights of products are consistent with peak 1 (FIG. 4(b)) being conjugated with a single molecule of MDA and peak 2 (FIG. 4(b)) being conjugated to a dimeric form of MDA.

Antigen Capture ELISA

Antibody specific against an antigen is coated onto an ELISA plate and used to capture antigen from patient plasma—either total plasma or LDL fractions are used. Binding is then detected using a second antibody specific against the antigen followed by an enzyme-conjugated anti-immunoglobulin for colorimetric detection.

Competitive ELISA

An ELISA plate is coated with either MDA-conjugated ALQYKLEGTTR (SEQ ID NO: 1) peptides or with oxidised or conjugated LDL or peptides of the same which will not be taken up by the high affinity LDL receptor. Serial dilutions of patient serum (for example total plasma or LDL fractions can be added) are added together with antibody of fixed dilution. Binding of antibody to coating antigen is detected using enzyme-conjugated anti-immunoglobulin, the extent of binding reducing as the concentration of antigen in the plasma increases. Results are standardised by producing a standard curve using MDA-conjugated ALQYKLEGTTR (SEQ ID NO: 1) peptides.

Immunoturbidimetry

Latex beads are coated with antibody specific to MDA-conjugated ALQYKLEGTTR (SEQ ID NO: 1) peptides, and the beads mixed with patient sera. The aggregation of the beads due to antibody cross-linking in the presence of specific antigen is analysed in autoanalyzers using immunoturbidimetry detection systems.

The procedures described above are used in diagnosis, for example in ELISA, immunoturbidimetry or dip-stick assays and test kits.

### Example 2

Tryptic digestion and mass spectrometry was performed to determine whether MDA is conjugated to the lysine or arginine residue contained within the peptide corresponding to SEQ ID NO:1.

Materials and Methods

In order to identify whether the MDA is conjugated in peptide SEQ ID NO:1 hydrolysis of the peptide was carried out with 1% trypsin at pH 8.5. Trypsin hydrolyses lysine and arginine linkages, except when proline is attached to the carboxyl group of lysine or arginine or where MDA is bound to lysine. In peptide SEQ ID NO:1, lysine appears in the centre of the peptide. The two positions for attachment of MDA are to the lysine and to the terminal arginine (less likely). If the lysine amino group is unmodified the trypsin will hydrolyse the peptide and fragments would be produced and the peaks would be detected (time of flight mass spectrometry) at approximately 700 mass units.

Results

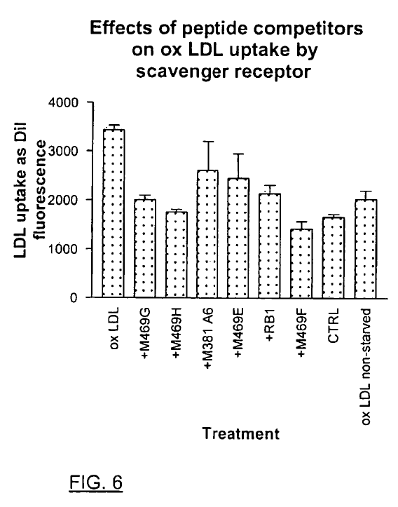

FIG. 5 shows the results of a mass spectrometry plot in which 2 distinct peaks are present—one at 1425 Da and the other at 2864.9 Da. The molecular weight of the conjugated peptide (i.e. the peptide conjugated to MDA) is predicted to be 1424 Da., compared with 1320 Da. for the predicted molecular weight of the native unconjugated peptide.

Thus, there are only two compounds present: the lysine-MDA conjugated peptide (1425 mass units) and its dimer (2867.9 mass units).

### CONCLUSION

The two possible points at which malondialdehyde (MDA) can react within the peptide corresponding to SEQ ID NO:1 are the lysine and arginine residues, both of which are basic amino acids. The protease trypsin hydrolyses lysine and arginine peptide linkages, except where proline is attached to the carboxyl group of lysine or arginine. Since there are no proline residues present within the peptide corresponding to SEQ ID NO:1, trypsin can cleave the peptide at either the lysine or the arginine residue. However, if MDA is present bound to the ε-amino group of lysine, the lysine peptide linkage hydrolysis by trypsin will be prevented, limiting the hydrolysis to arginine peptide linkages only. The mass spectrum produced after tryptic digestion of the peptide shows (FIG. 5) that the molecular weight of the peptide conjugated to MDA is 1424. The predicted molecular weight of the native unconjugated peptide is 1320 Da and the molecular weight of MDA is 72 Da, but when MDA conjugates to an amino acid it loses a water molecule, therefore only adding 54 Da to the peptide molecule per molecule of conjugated MDA. The difference between the two peaks on the mass spectrum is weight is approximately 104 Da, indicating that two molecules of MDA are conjugated per molecule of peptide.

If the MDA were conjugated to the arginine residue, the lysine residue would have been subject to enzymatic cleavage and the peptide would have been hydrolysed upon trypsin digestion, producing two peaks on the mass spectrum of approximate mass 700 Da. Although the spectrum shows that two peaks are produced (1425 and 2865 Da., respectively), these correspond to the non-digested peptide where MDA is conjugated to the lysine residue, and a dimeric form of this conjugated peptide.

In conclusion, MDA is conjugated to the lysine residue, and not the arginine residue.

### Example 3

An experiment was performed to determine in cell culture if uptake of oxidised LDL by U937 monocytes could be inhibited by the peptides according to SEQ ID NOs:1-3, both in their native state and conjugated to MDA.

Materials and Methods

LDL (1 mg/ml) was labelled with 1,1′-dioctadecyl-3,3,3′,3′,-tetramethylindocarbocyanine perchlorate (DiI) (300 μg/mg LDL) by incubation overnight at 37° C. in the dark under nitrogen. Excess DiI was removed by desalting into PBS (0.01 M phosphate buffer, 0.137 M sodium chloride, pH 7.4) through a PD-10 column.

U937 monocytes were maintained in RPMI media supplemented with heat inactivated fetal calf serum (10%) (FCS) and penicillin/streptomycin (1%). Cells were ‘starved’ of lipids by culturing in RMPI supplemented with penicillin/streptomycin (1%) in the absence of serum, and plated at 1 million cells per well in 6 well plates, 4 hours before treatment with DiI labelled LDL. In a starved state the cells exhibit greater lipid (cholesterol) uptake than non-starved cells, i.e. cells cultured in medium containing serum. Non-starved cells were also plated at 1 million cells per well in 6 well plates, 4 hours before treatment with DiI labelled LDL. Starved and non-starved cells were treated with DiI labelled LDL (oxidized) (50 μg) in the presence or absence of excess unlabelled peptide (native or MDA-conjugated) (500 μg) as competitor. LDL uptake was allowed for 20 hours before cells were washed in PBS (2×1 ml) and re-suspended in PBS-Triton-X-100 (5%) (200 μl) to lyse. Fluorescence was read at Ex 520 nm, Em 590 nm using a Molecular Devices Gemini XS microplate fluorimeter.

Results

Table 1 lists the codes used for peptides (native or MDA-conjugated) used as competitors of oxidised LDL uptake by the cells:

TABLE 1
Peptides used in inhibition of oxidised LDL uptake

| | |
|---|---|
| RB1 | peptide SEQ ID NO: 1 MDA-conjugated |
| M381 A6 | multiple antigenic peptide SEQ ID NO: 1 MDA-conjugated |
| | (mapped peptide) |
| M469F | peptide SEQ ID NO: 2 |
| M469E | peptide SEQ ID NO: 2 MDA-conjugated |
| M469H | peptide SEQ ID NO: 3 |
| M449G | peptide SEQ ID NO: 3 MDA-conjugated |

The results listed in Table 2 below correspond to one experiment, performed in triplicate and which have been reproduced in further experiments. Oxidised LDL corresponds to the amount of LDL taken up by the native receptor combined with the amount taken up by the scavenger receptor. Control corresponds to the uptake of LDL (i.e native, or non-oxidised LDL) by the native receptor alone (not the scavenger receptor), which will happen in any of the conditions shown. Note that Oxidised LDL(n/s) corresponds to the amount of LDL taken up by the native receptor combined with the amount taken up by the scavenger receptor, in conditions where the cells were cultured prior to treatment in medium containing serum, i.e. where the cells were non-starved.

TABLE 2
Inhibition of oxidised LDL uptake by
peptides in monocytes in cell culture

| Identity | Mean | SD | n | P versus Ox. LDL |
|---|---|---|---|---|
| Oxidised LDL | 3433 | 1656 | 3 | |
| Oxidised LDL + RB1 | 2130 | 301 | 3 | <0.01 |
| Oxidised LDL + M381 A6 | 2613 | 1016 | 3 | <0.01 |
| Oxidised LDL + M469F | 1414 | 268 | 3 | <0.001 |
| Oxidised LDL + M469E | 2447 | 867 | 3 | <0.05 |
| Oxidised LDL + M469H | 1756 | 97 | 3 | <0.001 |
| Oxidised LDL + M469G | 2005 | 160 | 3 | <0.01 |
| Control | 1657 | 102 | 3 | <0.001 |
| ox LDL non-starved | 2023 | 298 | 3 | <0.01 |

These results are plotted graphically in FIG. 6.

### CONCLUSION

In the uptake inhibition experiments, it is apparent that the peptides corresponding to SEQ ID NOs:1-3 can inhibit the uptake of oxidised LDL by U937 monocytes. The value associated with the Oxidised LDL (column 1, FIG. 6) corresponds to the amount of LDL taken up by the native receptor combined with the amount taken up by the scavenger receptor, and thus represents the maximum uptake value by the lipid starved monocytes. Note that the non-starved cells, which do not lack lipid, exhibit a correspondingly lower uptake value (column 9, FIG. 6). The Control sample (column 8, Figure B) corresponds to uptake of non-oxidised LDL by the native receptor alone (not the scavenger receptor)which will happen in any of the conditions shown. This value represents the minimum uptake value by the cells, and thus represents the extent to which a competing peptide could inhibit the uptake of oxidised LDL by the cells. Thus, as a competing peptide moves towards complete inhibition of uptake of oxidised LDL by the cells, the closer the uptake value would be to that of the Control value.

The results show that each of the three peptides can inhibit uptake of oxidised LDL by monocytes (columns 2-7, FIG. 6). Analysis for uptake of oxidised LDL in the presence of the peptide corresponding to SEQ ID NO:1 conjugated with MDA (RB1; column 6, FIG. 6), when compared to uptake of oxidised LDL alone (column 1, FIG. 6) confirms that the inhibition is statistically significant (p=<0.01, ANOVA followed by the Tukey post test).

The experiments thus provide evidence that the peptide corresponding to SEQ ID NO:1 conjugated to MDA inhibits uptake of LDL or partially modified LDL by the scavenger LDL receptor in monocytes in cell culture.

1. A molecule comprising the sequence of SEQ. ID NO: 1 having lysine 5 conjugated with MDA, wherein the molecule inhibits uptake of LDL by the high affinity LDL receptor.

2. The molecule according to claim 1, wherein the molecule inhibits uptake of LDL modified by conjugation with MDA.

3. The molecule according to claim 1, wherein the molecule is immunogen.

4. The molecule comprising SEQ. ID NO: 1 wherein lysine 5 is conjugated with MDA.

5. The molecule according to claim 3 wherein the molecule inhibits uptake of oxidised LDL by monocytes in cell culture.
